(19) United States
(12) Patent Application Publication
(10) Pub. No.: US 2004/0249288 A1
(43) Pub. Date: Dec. 9, 2004

(54) Ultrasonic puncture needle

(75) Inventor: Yusuke Ichikawa, Tokyo (JP)

Correspondence Address: SCULLY SCOTT MURPHY & PRESSER, PC, 400 GARDEN CITY PLAZA, GARDEN CITY, NY, 11530

(73) Assignee: OLYMPUS CORPORATION, TOKYO (JP)

(21) Appl. No.: 10/727,944

(22) Filed: Dec. 4, 2003

(30) Foreign Application Priority Data
Dec. 5, 2002 (JP) 2002-354289

(51) Int. Cl. A61B 8/14

(52) U.S. Cl. 600/464

(57) ABSTRACT

An ultrasonic puncture needle according to the present invention comprises a sheath for being inserted into a treatment tool insertion channel of an ultrasonic endoscope, and a needle tube for being inserted into tissue within the body cavity through the sheath, which includes multiple staggered-array doughnut-shaped recesses over a predetermined range on the surface of the tip portion of the needle tube from the portion near the tip thereof.

This application claims benefit of Japanese Application No. 2002-354289 filed on Dec. 5, 2002, the contents of which are incorporated by this reference.

### BACKGROUND OF THE INVENTION

1. Field of the Invention

The present invention relates to an ultrasonic puncture needle used by being inserted into the body cavity for performing suctioning biopsy, performing an injection, or the like.

2. Description of the Related Art

Conventionally, methods are known wherein puncture needle pierces body tissue so as to reach an affected portion within the body cavity, which is to be examined under ultrasonic observation, so as to sample tissue within the body cavity or body fluid. In these methods, the aforementioned puncture needle is inserted into the portion which is to be examined, e.g., digestive organ walls such as the stomach, duodenum, or the like, and furthermore, deep internal organs such as the spleen, liver, kidney, or the like, while observing the body cavity using an ultrasonic endoscope.

With these methods, the puncture needle is required to be inserted with a suitable insertion depth. That is to say, in the event that the puncture needle is inserted with too great a depth or too small a depth, the tip of the puncture needle does not stop at the portion which is to be examine. In this case, sampling of tissue within the body cavity, body liquid, or the like, at a desired portion cannot be performed.

With conventionally-used ultrasonic puncture needles (which will be referred to as “puncture needles” hereafter), the ultrasonic images of the puncture needle, generated from ultrasonic waves reflected from the surface of the puncture needle, cannot be clearly displayed in ultrasonic observation images. The reason is that the puncture needle is formed with an outer diameter of less than 1 mm, and accordingly, sufficient reflection echoes do not occur due to reflection from the surface of the puncture needle, leading to unclear ultrasonic images of the puncture needle having a low contrast in ultrasonic observation images. Accordingly, the surgeon cannot obtain the precise information with regard to the position of the tip of the puncture needle as to the portion which is to be examined, and the distance therebetween.

In order to solve the above-described problem, an arrangement has been disclosed in Japanese Unexamined Patent Application Publication No. 2003-190179, which includes a needle tube 30 in the shape of a pipe having a configuration as shown in FIGS. 1A and 1B. The needle tube 30 includes multiple staggered-array doughnut-shaped recesses 32 formed at predetermined positions on the surface of the tip portion 31 thereof. Thus, in the event that the ultrasonic waves are cast onto the needle tube 30 from directions perpendicular to the longitudinal direction of the needle tube 30, or even in the event the ultrasonic waves are cast from directions other than the perpendicular directions, the ultrasonic waves are reflected with great intensity in the incident direction, and thus the images of the needle tube are clearly displayed in the ultrasonic observation images.

### SUMMARY OF THE INVENTION

An ultrasonic puncture needle according to the present invention comprises a sheath which is inserted into a treatment tool insertion channel of an ultrasonic endoscope, and a needle tube for being inserted into tissue within the body cavity through the sheath, which included multiple staggered-array doughnut-shaped recesses over a predetermined range on the surface of the tip portion of the needle tube from the portion near the tip of the needle tube. Accordingly, a needle tube image with small-sized unimaged portion is generated in an ultrasonic observation image. Thus, the surgeon can perform insertion of the puncture needle into the portion which is to be examined, in a sure manner.

The above and other objects, features and advantages of the invention will become more clearly understood from the following description referring to the accompanying drawings.

### BRIEF DESCRIPTION OF THE DRAWINGS

FIG. 1A is a side view for describing the configuration of the tip portion of a conventional needle tube;

FIG. 1B is a bottom view for describing the configuration of the tip portion of a conventional needle tube;

FIG. 2 is a diagram for describing an ultrasonic puncture needle;

FIG. 3A is a side view of the tip portion of a needle tube according to the present invention;

FIG. 3B is a top view of the tip portion of the needle tube according to the present invention;

FIG. 3C is a bottom view of the tip portion of the needle tube according to the present invention;

FIG. 4 is a schematic drawing showing the needle tube in an unfurled manner, for describing an array pattern of doughnut-shaped recesses included on the tip portion of the needle tube;

FIG. 5 is a cross-sectional view taken along line 4-4 in FIG. 3A;

FIG. 6A is a diagram which shows the positional relation between the needle tube and an ultrasonic endoscope, wherein the back of a cutting-tip portion of the needle tube faces the scanning face of an ultrasonic transducer, at the time of the surgeon protruding the needle tube of the ultrasonic puncture needle from a treatment tool insertion channel of the ultrasonic endoscope;

FIG. 6B is a diagram which shows an ultrasonic image generated in the positional relation shown in FIG. 6A;

FIG. 7A is a diagram which shows the positional relation between the needle tube and the ultrasonic endoscope, wherein the side of the cutting-tip portion of the needle tube faces the scanning face of the ultrasonic transducer, at the time of the surgeon protruding the needle tube of the ultrasonic puncture needle from the treatment tool insertion channel of the ultrasonic endoscope;

FIG. 7B is a diagram which shows an ultrasonic image generated in the positional relation shown in FIG. 7A;

FIG. 8A is a diagram which shows the positional relation between the needle tube and the ultrasonic endoscope, wherein the cutting-tip portion of the needle tube faces the scanning face of the ultrasonic transducer, at the time of the surgeon protruding the needle tube of the ultrasonic puncture needle from the treatment tool insertion channel of the ultrasonic endoscope; and

FIG. 8B is a diagram which shows an ultrasonic image generated in the positional relation shown in FIG. 8A.

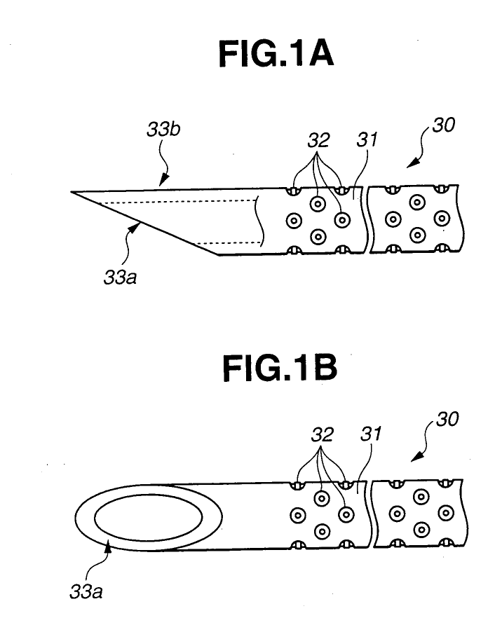

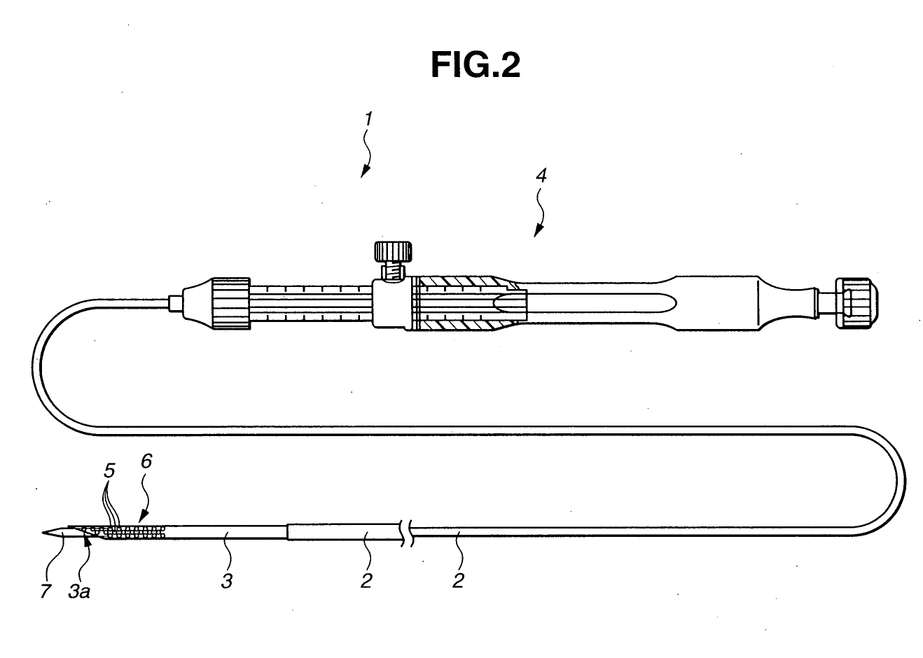

### DETAILED DESCRIPTION OF THE PREFERRED EMBODIMENTS

Description will be made below regarding embodiments of the present invention with reference to the drawings.

As shown in the drawings, an ultrasonic puncture needle 1 according to the present embodiment principally comprises a sheath 2, a needle tube 3, and an operating portion 4.

The aforementioned sheath 2 is inserted into a treatment tool insertion channel of an endoscope, for example. The aforementioned operating portion 4 serves as a holding portion, as well, and is disposed at the base portion of the aforementioned sheath 2. The needle tube 3 is formed of a slender stainless pipe with a small tube thickness, for example. The needle tube 3 includes a puncturing portion 6 having a sharp cutting-tip portion 3a or the like, at the tip thereof. The needle tube 3 is slidably disposed within the sheath 2 through the operating portion 4.

Note that an arrangement may be made wherein a stylette 7 having a sharp-shaped tip is detachably disposed within the through hole of the aforementioned needle tube 3.

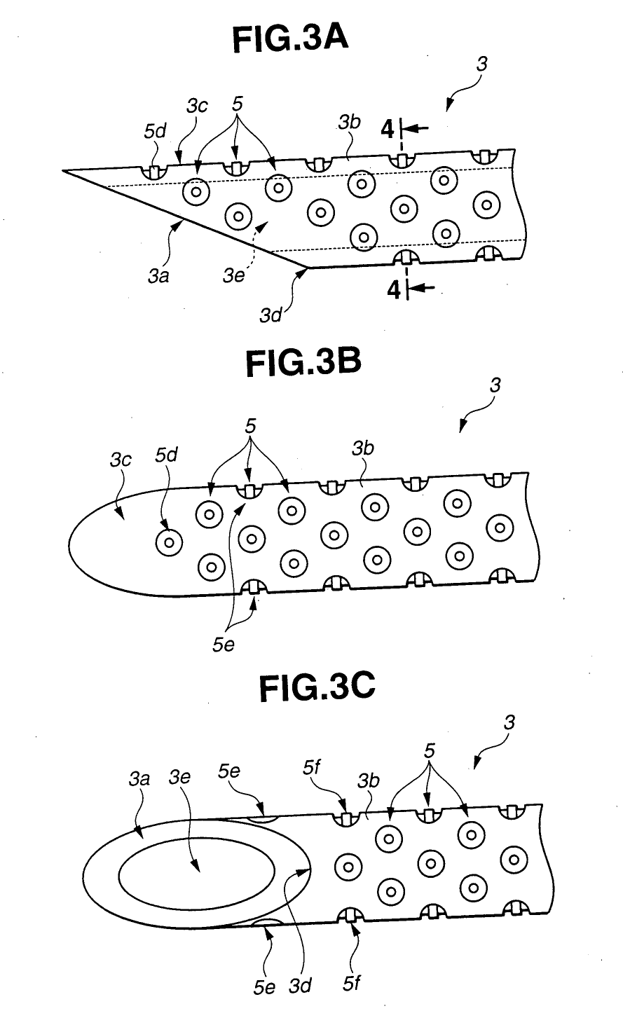

Next, description will be made regarding the tip portion of the needle tube 3 with reference to FIGS. 3A through 5.

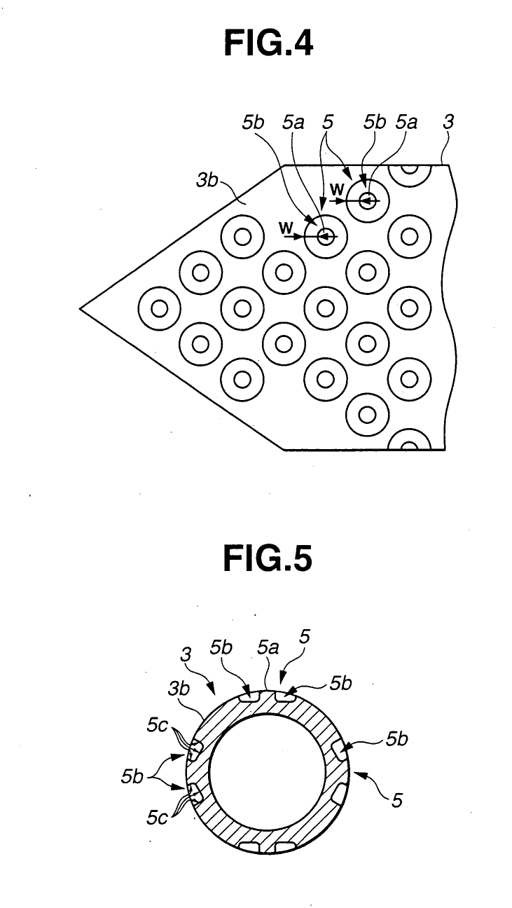

As shown in FIGS. 3A through 4, a tube portion 3b forming the puncturing portion 6 provided in a predetermined range from the portion near the tip of the aforementioned needle tube 3 includes multiple staggered-array doughnut-shaped recesses 5 on the surface thereof, serving as so-called ultrasonic wave reflection means for reflecting ultrasonic waves. The multiple doughnut-shaped recesses 5 are formed so as to not have adverse effects upon the aforementioned cutting-tip portion 3a. These multiple doughnut-shaped recesses 5 are formed on the tip side of the tube portion 3b with a great density from a predetermined portion on a back-of-cutting-tip portion 3c on the back side of the aforementioned cutting-tip portion 3a so as to be formed on the tube portion 3b in a radial pattern. The aforementioned multiple staggered-array doughnut-shaped recesses 5 are formed on the tube portion 3b using a YAG laser apparatus or an electric discharge machining apparatus under predetermined control set so as to form a staggered-array pattern.

As shown in FIG. 5, the aforementioned doughnut-shaped recesses 5 are formed with the faces 5c of the bottoms and the sides, each generally flat in the cross-sectional view thereof, so as to obtain reflection echoes with a great intensity for the incident ultrasonic waves with a shallow incident angle or the like. These doughnut-shaped recesses 5 are formed using the YAG laser with a laser beam spot diameter set to 0.1 mm under positioning control for the laser beam and the needle tube 3, for example. Specifically, with regard to each doughnut-shaped recess, a ring-shaped recess 5b with a predetermined width (W) formed at a predetermined position on the needle tube 3, whereby the center portion thereof remain as protrusion 5a with a predetermined size, as shown in FIGS. 4 and 5.

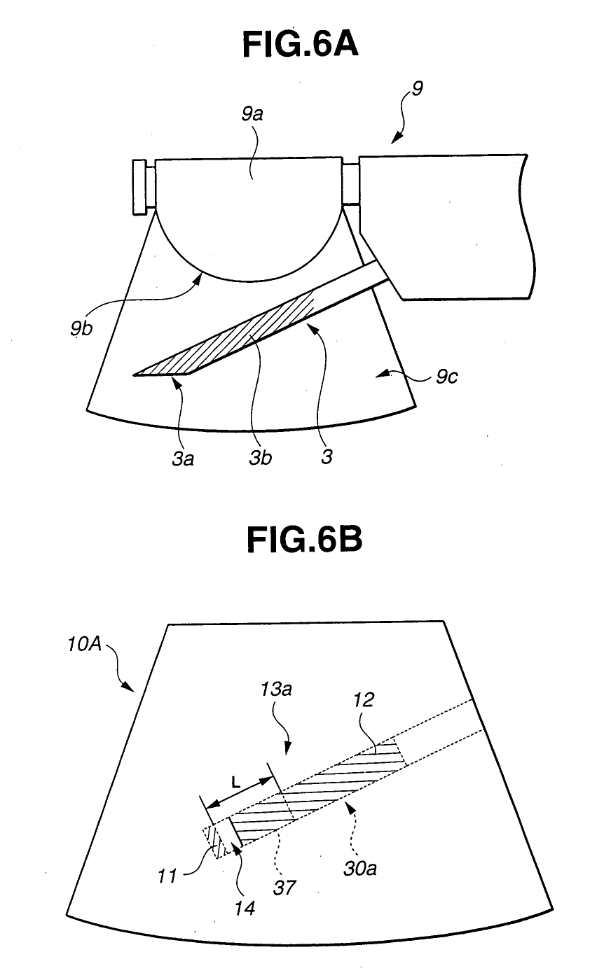

Next, description will be made regarding the operation of the ultrasonic puncture needle 1 having the above-described configuration in a case of the surgeon protruding the needle tube 3 thereof from the treatment tool insertion channel of the ultrasonic endoscope, with reference to FIGS. 6A through 8B.

First, as shown in FIG. 6A, the needle tube 3 is protruded from an unshown treatment tool insertion channel of an ultrasonic endoscope 9 so as to be positioned within the ultrasonic scanning range 9c of an ultrasonic transducer 9a. Now, let us say that the back-of-cutting-tip portion 3c of the needle tube 3 faces in the direction of the ultrasonic transducer 9a. In this case, as shown in the top view in FIG. 3b, the doughnut-shaped recesses 5 formed on the tube portion 3b in the range from the doughnut-shaped recess 5 at the base thereof up to the first doughnut-shaped recess 5d at the tip thereof face the scanning face 9b of the ultrasonic transducer 9a.

Thus, as shown in FIG. 6B, a needle tube image 13a is displayed in an ultrasonic observation image 10A. The needle tube image 13a contains a needle-tube-tip image 11 due to the ultrasonic waves reflected from the tip of the cutting-tip portion 3a and a needle-tube-surface image 12 representing the tube portion 3b due to the ultrasonic waves reflected from the doughnut-shaped recesses 5 formed on the back-of-cutting-tip portion 3c of the needle tube 3 in a predetermined range from the portion near the tip thereof.

The aforementioned needle tube image 13a forms an ultrasonic image representing the generally entire needle tube from the tube portion 3b including the doughnut-shaped recesses 5 up to the tip of the needle tube. However, more precisely, an unimaged portion 14 occurs between the needle-tube-tip image 11 and the needle-tube-surface image 12. The aforementioned unimaged portion 14 occurs due to the portion having no doughnut-shaped recesses between the tip of the needle tube and the first doughnut-shaped recess 5d formed at the head thereof shown in the top view in FIG. 3B. However, the unimaged portion 14 occurs with a length markedly shorter than the length L of an unimaged portion 37 occurring in a needle tube image 30a generated due to the conventional needle tube 30 denoted by broken lines.

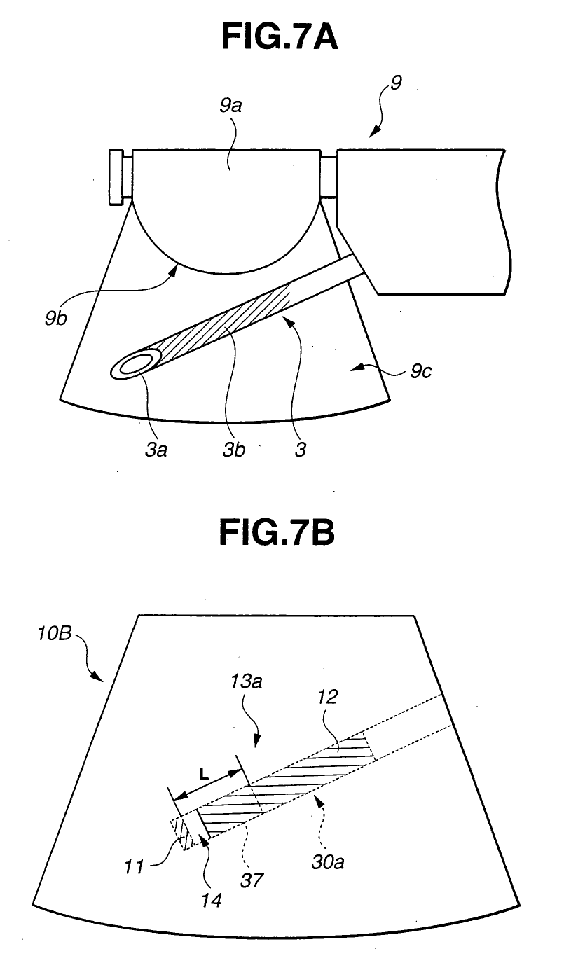

Next, the needle tube 3 is protruded from the unshown treatment tool insertion channel of the ultrasonic endoscope 9 so as to be positioned within the ultrasonic scanning range 9c of the ultrasonic transducer 9a as shown in FIG. 7A. Now, let us say that one side of the cutting-tip portion 3a of the needle tube 3 faces in the direction of the aforementioned ultrasonic transducer 9a. In this case, the doughnut-shaped recesses 5, formed on the tube portion 3b, including the sides of the doughnut-shaped recesses 5 in the range from the doughnut-shaped recess 5 at the base of the tube portion 3b up to the first doughnut-shaped recess 5d at the head thereof, face the scanning face 9b of the aforementioned ultrasonic transducer 9a, as shown in the side view in FIG. 3A.

Accordingly, while the aforementioned scanning face 9b and the doughnut-shaped recesses 5 face each other in a manner somewhat different from the relation in the above-described case shown in FIG. 6A, the doughnut-shaped recesses 5 in the range from the doughnut-shaped recess 5 at the base of the needle tube 3 up to the first doughnut-shaped recess 5d at the head thereof face the scanning face 9b generally in the same way.

Thus, as shown in FIG. 7B, the needle-tube image 13a is displayed in an ultrasonic observation image 10B, which contains the needle-tube-tip image 11 and the needle-tube-surface image 12 with the aforementioned unimaged portion 14 therebetween, generally in the same way as shown in FIG. 6B described above. The unimaged portion 14 occurs with a length markedly shorter than the length L of the unimaged portion 37 occurring in the needle tube image 30a generated due to the conventional needle tube 30 denoted by broken lines.

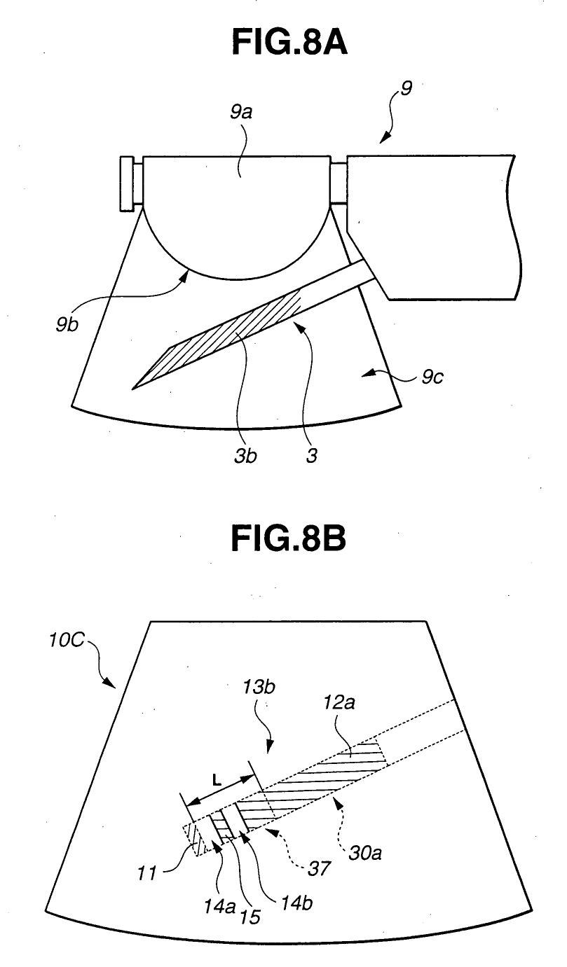

Next, the needle tube 3 is protruded from the unshown treatment tool insertion channel of the ultrasonic endoscope 9 so as to be positioned within the ultrasonic scanning range 9c of the ultrasonic transducer 9a as shown in FIG. 8A. Now, let us say that the cutting-tip portion 3a of the needle tube 3 faces the aforementioned ultrasonic transducer 9a. In this case, the cutting-tip portion 3a formed of an inclined face having a through hole 3e, the sides of doughnut-shaped recesses 5e formed on the side face of the cutting-tip portion 3a, and the tube portion 3b including the doughnut-shaped recesses 5 formed on the rear side of the base of the cutting-tip portion 3a, face the scanning face 9b of the ultrasonic transducer 9a, as shown in the bottom view in FIG. 3C.

Thus, a needle-tube image 13b is displayed in an ultrasonic observation image 10C as shown in FIG. 8B. The needle tube image 13b contains the needle-tube-tip image 11 generated due to reflection of the ultrasonic waves from the tip of the cutting-tip portion 3a, a needle-tube-rear-portion image 15 generated due to reflection of the ultrasonic waves from an edge rear portion 3d of the cutting-tip portion 3a, and a needle-tube-surface image 12a generated due to reflection of the ultrasonic waves from the multiple doughnut-shaped recesses 5 formed on the rear side of the cutting-tip portion 3a.

While the aforementioned needle tube image 13b is an ultrasonic image which represents the generally entire needle tube in the range from the tube portion 3b including the doughnut-shaped recesses 5 up to the tip of the needle tube, an unimaged portion 14a occurs between the needle-tube-tip image 11 and the needle-tube-rear-portion image 15, as well as unimaged portion 14b occurring between the needle-tube-rear-portion image 15 and the needle-tube-surface image 12.

The aforementioned unimaged portion 14a occurs due to the portion between the tip of the needle portion 3a and the edge rear portion 3d, shown in the bottom view in FIG. 3C. On the other hand, the aforementioned unimaged portion 14b occurs due to the portion between the edge rear portion 3d and doughnut-shaped recesses 5f formed on the rear side of the cutting-tip portion 3a, shown in the bottom view in FIG. 3C. While such unimaged portions 14a and 14b occur in the needle tube image 13b, these unimaged portions 14a and 14b occurs with lengths markedly shorter than the length L of the unimaged portion 37 occurring in the needle tube image 30a generated due to the conventional needle tube 30 denoted by broken lines.

As described above, with the ultrasonic puncture needle according to the present invention, the multiple staggered-array doughnut-shaped recesses are formed so as to be spread with a great density over a predetermined range in a radial pattern from the portion near the tip of the needle tube, and accordingly, the ultrasonic waves output from the ultrasonic transducer are reflected from the needle tube with a greater intensity and free of large portions where the ultrasonic waves are not reflected, thereby displaying a clear needle-tube image without large-sized unimaged portions on an ultrasonic observation image.

Furthermore, with the ultrasonic puncture needle according to the present invention, the doughnut-shaped recesses are formed with a great density in a predetermined pattern such that the doughnut-shaped recesses do not have adverse effects upon the insertion performance of the needle tube.

Thus, with the ultrasonic puncture needle according to the present invention, the surgeon can perform precise measurement of the positioning relation and the distance between the needle tube and the portion which is to be examined, and also can perform insertion of the needle tube in a sure manner, even in the event that the surgeon performs insertion of the needle tube into a small-sized affected portion near the wall of the body cavity.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An ultrasonic puncture needle comprising:
a sheath which is inserted into a treatment tool insertion channel of an ultrasonic endoscope; and
a needle tube for being inserted into tissue within the body cavity through the sheath, which includes a plurality of staggered-array doughnut-shaped recesses over a predetermined range on the surface of the tip portion of the needle tube from the portion near the tip of the needle tube.

2. An ultrasonic puncture needle according to claim 1, wherein the plurality of doughnut-shaped recesses are arrayed so as to be spread in a radial pattern from the tip of the needle tube.

3. An ultrasonic puncture needle according to claim 1, wherein the multiple doughnut-shaped recesses are formed using a laser apparatus or an electric discharge machining apparatus.

4. An ultrasonic puncture needle according to claim 3, wherein the multiple doughnut-shaped recesses are formed using a laser apparatus or an electric discharge machining apparatus under positioning control set so that the doughnut-shaped recesses have no adverse effects on an cutting-tip portion forming the needle tube due to overlap of the doughnut-shaped recesses and the cutting-tip portion.

5. An ultrasonic puncture needle comprising a needle tube which is to be inserted into a treatment tool insertion channel of an ultrasonic endoscope so as to be inserted into tissue within the body cavity, wherein the needle tube includes a plurality of recesses over a predetermined range on the surface of the tip portion thereof from the tip thereof on the back side of an cutting-tip portion.

6. An ultrasonic puncture needle according to claim 5, wherein the plurality of recesses are arrayed so as to be spread in a radial pattern from the tip of the needle tube.

7. An ultrasonic puncture needle according to claim 5, wherein the plurality of recesses are formed at positions such that overlap of the recesses and the cutting-tip portion does not occur.

8. An ultrasonic puncture needle according to claim 6, wherein the plurality of recesses are formed at positions such that overlap of the recesses and the cutting-tip portion does not occur.

9. An ultrasonic puncture needle according to claim 5, wherein the plurality of recesses are formed in a doughnut shape using a laser apparatus or an electric discharge machining apparatus.

10. An ultrasonic puncture needle according to claim 6, wherein the plurality of recesses are formed in a doughnut shape using a laser apparatus or an electric discharge machining apparatus.

11. An ultrasonic puncture needle according to claim 7, wherein the plurality of recesses are formed in a doughnut shape using a laser apparatus or an electric discharge machining apparatus.

12. An ultrasonic puncture needle comprising:
a puncturing portion formed with a suitable length at the tip of the ultrasonic puncture needle; and
a tube portion formed in the shape of a tube at the rear end of the puncturing portion, wherein the puncturing portion is formed of an cutting-tip portion and a tube-shaped portion formed as an extension of the tube portion, which includes ultrasonic-reflection means on the surface of the tip portion thereof.

13. An ultrasonic puncture needle according to claim 12, wherein the ultrasonic-reflection means comprises a plurality of doughnut-shaped recesses formed and arrayed so as to be spread over a predetermined range on the surface of the tip portion in a radial pattern from the tip of the tube portion on the back side of the cutting-tip portion.

14. An ultrasonic puncture needle according to claim 13, wherein the plurality of doughnut-shaped recesses are formed using a laser apparatus or an electric discharge machining apparatus.

15. An ultrasonic puncture needle according to claim 14, wherein the plurality of doughnut-shaped recesses are formed at positions such that overlap of the recesses and the cutting-tip portion forming the needle tube does not occur, using a laser apparatus or an electric discharge machining apparatus.

16. An ultrasonic puncture needle according to claim 12, wherein the ultrasonic-reflection means comprises a plurality of recessed portions formed and arrayed so as to be spread in a predetermined range on the surface of the tip portion in a radial pattern from the tip of the tube portion on the back side of the cutting-tip portion.

17. An ultrasonic puncture needle according to claim 16, wherein the plurality of recessed portions are formed at positions such that overlap of the recessed portions and the cutting-tip portion does not occur.

18. An ultrasonic puncture needle according to claim 16, wherein the plurality of recessed portions are formed in a doughnut shape using a laser apparatus or an electric discharge machining apparatus.

19. An ultrasonic puncture needle according to claim 17, wherein the plurality of recessed portions are formed in a doughnut shape using a laser apparatus or an electric discharge machining apparatus.
